(19) United States
(12) Patent Application Publication
(10) Pub. No.: US 2006/0177489 A1
(43) Pub. Date: Aug. 10, 2006

(54) Attachment of chitosan to surfaces using rehydration process

(76) Inventors: Debora Flanagan Massouda, Wilmington, DE (US); Patricia Metzger Cotts, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/349,485

(22) Filed: Feb. 7, 2006

Related U.S. Application Data
(60) Provisional application No. 60/650,654, filed on Feb. 7, 2005.

(51) Int. Cl. A61K 9/70; B32B 5/02; B32B 15/00; C08B 37/08

(52) U.S. Cl. 424/443; 536/20; 442/152; 428/615

(57) ABSTRACT

A method for attaching chitosan to the surface of polymers that includes at least one rehydration step has been developed to provide more effective and stable chitosan coating. Polymers produced using this method and articles that are made with these polymers provide antibacterial and anti-odor properties.

This application claims priority to U.S. provisional application Ser. No. 60/650654, filed Feb. 7, 2005, the entire disclosure of which is incorporated herein by reference.

This invention relates to the field of antimicrobial materials. Specifically, a method is provided for treating a surface with chitosan such that attachment is improved.

### BACKGROUND OF THE INVENTION

As evidenced by the presence in the market of numerous materials for eliminating or minimizing human contact with bacteria, there is clearly a demand for materials and/or processes that either minimize or kill bacteria encountered in the environment. Such materials are useful in connection with food packaging, preparation, and handling as well as in areas of personal hygiene, such as in garments and personal care articles, and in locations with high potential for microbial contamination such as bathrooms. Similarly, antibacterial materials are useful in hospitals and nursing homes where people with lowered resistance are especially vulnerable to bacteria.

Chitosan compounds are known to provide antimicrobial activity as bacteriocides and fungicides (see, e.g., T. L. Vigo, “Antimicrobial Polymers and Fibers: Retrospective and Prospective,” in Bioactive Fibers and Polymers, J. V. Edwards and T. L. Vigo, eds., ACS Symposium Series 792, pp. 175-200, American Chemical Society, 2001). Chitosan is also known to impart antiviral activity, though the mechanism is not yet well understood (see, e.g., Chirkov, Applied Biochemistry and Microbiology (Translation of Prikladnaya Biokhimiya i Mikrobiologiya) (2002), 38(1), 1-8). Additionally, chitosan is known to impart anti-odor properties (see, for example, WO 1999061079(A1)).

Chitosan is the commonly used name for poly-[1-4]-β-D-glucosamine. Chitosan is chemically derived from chitin which is a poly-[1-4]-β-N-acetyl-D-glucosamine, which, in turn, is derived from the cell walls of fungi, the shells of insects and, especially, crustaceans. Thus, it is inexpensively derived from widely available materials. It is available as an article of commerce from, for example, Primex Corporation (Norway), Biopolymer Engineering, Inc. (St. Paul, Minn.), Biopolymer Technologies, Inc. (Westborough, Mass.), and CarboMer, Inc. (Westborough, Mass.).

There are multiple known methods of treating materials with chitosan to prepare antimicrobial articles. Chitosan treatment of materials may include crosslinking or generation of reactive groups to attach chitosan to the material surface. In U.S. Pat. No. 6,197,322, chitosan is coated onto hydrophobic materials, which are found to have increased antimicrobial activity. In the method for preparing the chitosan-treated hydrophobic material, the chitosan is crosslinked. In co-owned, co-pending U.S. Patent Application No. 2003/0091612, polyolefin articles are treated with an aqueous mixture of chromic acid and sulfuric acid, washed with deionized water, soaked in concentrated nitric acid, and again washed with deionized water before treatment with chitosan solution. In co-owned, co-pending U.S. Patent Application Ser. No. 60/496296, a polymer surface that contains amino-reactive functional groups is treated with a chitosan solution to produce an antimicrobial polymeric material.

Chitosan may also be prepared for uses other than as a surface treatment. In U.S. Pat. No. 5,599,916, chitosan salt is treated under humid conditions to produce swellable and water-insoluble chitosan salt with increased ability to absorb liquid for use in personal care absorbent products. No coating or surface treatment is described or suggested to be possible.

While antimicrobial articles are made by the above methods, a simpler, more economical and more effective process of coating surfaces with chitosan to provide antimicrobial properties is desirable.

### SUMMARY OF THE INVENTION

The present invention provides a method of attaching chitosan to the surface of polymers. Also disclosed are polymers coated with chitosan using said method, and articles comprising said polymers.

One aspect is for a method for attaching chitosan to a polymer comprising:
(a) providing a wettable surface of a polymer;
(b) contacting a chitosan acid salt solution comprising chitosan and at least one organic acid to the wettable surface of (a) to produce a chitosan-coated polymer;
(c) drying the chitosan-coated polymer produced in (b);
(d) rehydrating the dried chitosan-coated polymer of (c); and
(e) drying the chitosan-coated polymer of (d).

Preferred organic acids include, for example, acetic acid, formic acid, butyric acid, proprionic acid, valeric acid, citric acid, and mixtures thereof.

In a preferred embodiment, the chitosan acid salt solution comprises the at least one organic acid in a stoichiometric amount with respect to the concentration of the chitosan. Chitosan is preferably present in the chitosan acid salt solution in a range of from about 0.1% to about 10%, more preferably in a range of from about 2% to about 10%, and most preferably at about 4%.

Optionally, in another aspect, the method above comprises before step (a) the further step of pretreating a nonwettable surface on the polymer to produce a wettable surface on the polymer. Pretreating can be performed by, for example, corona treatment, plasma treatment, electrical discharge, acid etching, or chemical treatment.

### BRIEF DESCRIPTION OF THE FIGURES

FIG. 1 shows light scattering, viscosity, and refractive index chromatograms of a 4% chitosan solution in 2% acetic acid that was held at 50° C. for two weeks.

### DETAILED DESCRIPTION OF THE INVENTION

The entire content of all cited references is incorporated herein by reference.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The present invention provides a method of applying chitosan to polymers that results in enhanced attachment of chitosan to the surface of the polymer leading to improved stability of the chitosan coating. Using a method of the invention, chitosan is attached with improved stability to amino-reactive surfaces. Also, using a method of the invention chitosan is stably attached to un-primed, inert surfaces. In a method of the invention, following the application of a chitosan coating to a polymer in chitosan solution, the polymer with a chitosan coating is rehydrated and dried causing a more stable attachment of chitosan onto the polymer surface. A chitosan coating on polymers provides an antimicrobial and anti-odor property to these polymers. Enhanced attachment increases the antimicrobial activity and also improves the stability of the antimicrobial and anti-odor activity.

The present invention also is directed to antimicrobial and anti-odor polymer produced using the method of the invention and to articles comprising same. Articles comprising polymers treated by a method of the invention exhibit antibacterial functionality wherein bacterial growth is reduced as the article is commonly used. Antimicrobial functionality may also be provided, wherein other microbes in addition to bacteria, such as fungi and viruses, have reduced growth when in contact with articles of the invention.

The following definitions and abbreviations aid in the interpretation of the claims and the specification.

The term “antibacterial,” as used herein, means bactericidal as is commonly known in the art. The number of bacteria present after contact with an antibacterial material is substantially reduced from the number initially present. The number of bacteria present is normally measured as colony forming units.

The term “antimicrobial,” as used herein, means antibacterial as well as having fungicidal and antiviral activities as is commonly known in the art.

“Amino-reactive groups” as used herein refers to chemical functionalities that readily undergo chemical reaction with an NH₂ group. Examples include positively charged species such as metal ions, anhydrides, carboxylic acids, isocyanates, epoxides, acid chlorides, and enones.

The phrase “polymer comprises amino-reactive functional groups as polymerized” as used herein refers to homopolymers and copolymers (including graft copolymers) which, as (co)polymerized, present a surface containing amino-reactive functional groups in sufficient quantity that the amino groups of the chitosan agent react with the substrate's surface to form a stable coating without the need for additional chemical or physical modification or priming of the substrate's surface (for example, treatment with caustic, acid, or plasma etching).

The term “surface” refers to the outer or topmost boundary of a material. Types of surfaces include properties such as being flat and solid such as of a film, fibrous as in woven knit or nonwoven fabric, porous as in a filter, rough, or permeable.

Polymers for Chitosan Treatment

Polymers that have inert surfaces without reactive groups as well as those that do have reactive groups may be used in the method of the invention.

Polyolefins without reactive groups that are suitable for use in the present invention include, but are not limited to, olefinic homopolymers such as polypropylene and polyethylene, including such polyethylenes as low density polyethylene (LDPE), very low density polyethylene (VLDPE), linear low density polyethylene (LLDPE), high density polyethylene (HDPE), ultra low density polyethylene (ULDPE), metallocene-catalyzed polyethylene, high performance polyethylene (HPPE), and ultra high molecular weight polyethylene (UHMWPE). Additional polymers with inert surfaces may be used in the method of the invention including, but not limited to, polyesters, nylons, and fluoropolymers.

Polymers with reactive groups suitable for the present invention include graft copolymers comprising a graft monomer and a backbone polymer, such as, but not limited to, those described in U.S. Pat. No. 4,026,967, in which the graft monomers include thermally stable unsaturated carboxylic anhydrides and dianhydrides, and the backbone polymers are preferably polymers of ethylene and copolymers derived from ethylene and C₃-C₈ alpha-olefins, including copolymers of at least one olefin with other monomers. Examples of suitable graft monomers for use in the present invention include methacrylic acid, acrylic acid, glycidyl methacrylate, 2-hydroxy ethylacrylate, 2-hydroxy ethyl methacrylate, diethyl maleate, monoethyl maleate, di-n-butyl maleate, maleic anhydride, maleic acid, fumaric acid, itaconic acid, itaconic anhydride, dodecenyl succinic anhydride, 5-norbornene-2,3-anhydride, and nadic anhydride (3,6-endomethylene-1,2,3,6-tetrahydrophthalic anhydride). Fumaric acid, maleic anhydride, and glycidyl methacrylate are particularly preferred graft monomers. Examples of suitable backbone polymers are polypropylene; polyethylene, e.g., high density polyethylene (HDPE), low density polyethylene (LDPE), linear low density polyethylene (LLDPE), metallocene-catalyzed polyethylene, very low density polyethylene (VLDPE), ultrahigh molecular weight polyethylene (UHMWPE), high performance polyethylene (HPPE); copolymers of ethylene and propylene; copolymers derived from ethylene or propylene and at least one monomer chosen from propylene, methyl acrylate, ethyl acrylate, n-butyl acrylate, methyl methacrylate, acrylic acid, methacrylic acid and carbon monoxide; and copolymers of olefins with a diolefin, such as a copolymer of ethylene, or of propylene, or of ethylene and other olefins, with: linear aliphatic nonconjugated dienes of at least six carbon atoms (such as 1,4-hexadiene) and other dienes, conjugated or not, such as norbornadiene, dicyclopentadiene, ethylidene norbornene, butadiene, and the like. One example of a commercially available graft copolymer suitable for use in the present invention is Bynel® 4033, a maleic anhydride grafted HDPE available from E. I. du Pont de Nemours and Company (Wilmington, Del., USA).

Another type of polymer suitable for use in the present invention is a copolymer of an olefin with vinyl esters such as vinyl acetate, with unsaturated acids or esters of those acids such as acrylic or methacrylic acid, or 1-8 carbon alkyl acrylates and methacrylates, or mixtures of these comonomers. Ethylene is the preferred olefin. An example of a commercially available material is Nucrel® ethylene acid copolymer resin available from E. I. du Pont de Nemours and Company (Wilmington, Del., USA).

Other polymers suitable for use in the present invention are ionomers. The term “ionomer” as used herein refers to a polymer with inorganic salt groups attached to the polymer chain (Encyclopedia of Polymer Science and Technology, 2nd ed., H. F. Mark and J. I. Kroschwitz eds., vol. 8, pp. 393-396). Two typical ionomer structures are shown below: 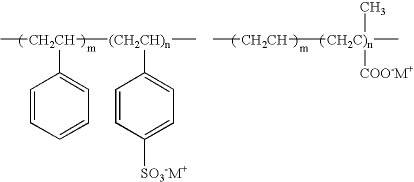
where the ratio of m to n is usually on the order of 10 to 100; that is, typically only about 1 to 9% of the repeat units contain ionic groups. Ions M are typically metal ions like lithium, sodium, or zinc but can be other cations, for example, ammonium. Typically, an acid form of the polymer is made first and then neutralized to the desired degree with base containing the desired metal ions. Partially neutralized poly(ethylene-co-methacrylic acid) and partially neutralized poly(ethylene-co-acrylic acid) are examples of ionomers, as is sulfonated polystyrene. Some examples of commercially available ionomers are Surlyn® thermoplastic resin and Nafion® perfluorinated sulfonic acid membranes, available from E. I. du Pont de Nemours and Company (Wilmington, Del., USA); Flemion® perfluorocarboxylate ionomers developed by Asahi Glass Company in Japan; and a sulfonated ethylene-propylene terpolymer from Exxon. Polyesters and polyamides that have been polymerized with a low level of sulfonated comonomer to enhance textile dyeability (see, e.g., U.S. Pat. Nos. 5,559,205; 5,607,765; and 3,389,549) and sulfonated aromatic polyamides (see, e.g., U.S. Pat. Nos. 3,567,632 and 4,595,708) such as those used in reverse osmosis membranes and other selective separation membranes are also suitable polymers for the present invention.

Examples of suitable polymer blends for use in the present invention include, but are not limited to, toughened grades of semicrystalline thermoplastics, such as toughened polyesters and polyamides, wherein the toughener is a polymer that contains amino-reactive groups as polymerized.

A method of the invention may be performed with any of the polymers described above and with articles that include any of these polymers.

Chitosan

Articles of the present invention have at least one layer of chitosan thereon. Chitosan is the common name for poly-[1-4]-β-D-glucosamine. Chitosan is chemically derived from chitin which is a poly-[1-4]-β-N-acetyl-D-glucosamine which, in turn, is derived from the cell walls of fungi, the shells of insects and, especially, crustaceans. Chitin is treated with strong alkalis to remove acetyl groups producing chitosan. Depending on the specific treatment of chitin, chitosan may vary in the degree of deacetylation. Chitosan is generally insoluble in water, but dissolves in dilute solutions of organic acids such as acetic, formic, tartaric, valeric, and proprionic acids. Preparations of unusually short chitosan polymers of low molecular weight, that is less than about 10,000 Daltons, are soluble in water. This type of preparation is uncommon and not used in the present invention. Typical chitosan preparations with varying molecular weights of individual species are used in the present invention.

Chitosan salts formed by the interaction of chitosan and an acid are suitable for the present invention. The acid may be an organic acid such as to form salts of chitosan, for example, chitosan acetate, chitosan formate, chitosan acrylate, chitosan butyrate, chitosan valerate, and chitosan proprionate. Mixtures of different chitosan salts are also suitable. Preferred salts for use in the invention are formed from volatile acids such as acetic acid and formic acid. Chitosan salts may have a wide range of molecular weights due to different chain lengths of the polymer. A chitosan salt preparation having a mixture of molecular weights is suitable for the present invention.

Chitosan solutions may be used in the method of the invention at various times after their preparation. Chitosan salt solutions form increasing, yet small, amounts of aggregates upon storage. Aggregate formation increases at cool (about 40° F. (about 4° C.)) and at hot (about 50° C.) temperatures over a period of two to three weeks of storage. Aggregate formation depends upon chitosan concentration, acid concentration, and temperature of storage. Aggregates formed in chitosan solutions may promote insolubility of a chitosan coating on a polymer during the rehydration process in a method of the invention. Thus, chitosan solutions may be aged prior to use in the method of the present invention. Ageing may be for a period of one week or longer, with preferred ageing of about six weeks.

In addition to promotion of insolubility by aggregates in chitosan solutions, anhydrous crystals in chitosan solutions may also promote insolubility. Anhydrous crystals may form in chitosan solutions (J. Kawanda et al., J. Carbohydr. Chem. 18(5): 559-71 (1999)) in the presence of factors such as elevated temperature, high acid, and shear forces. Chitosan solutions with aggregates or anhydrous crystals may be added to fresh chitosan solutions to provide insolubility-promoting agents.

Chitosan Treatment Strategy

Though chitosan is generally known to have antimicrobial properties, surfaces incubated with chitosan do not always acquire antimicrobial properties. For a polymer to acquire antimicrobial properties, methods disclosed herein allow the chitosan to maintain antimicrobial function while being retained on the surface of the polymer. A method of the present invention includes a rehydration treatment following contact of a polymer with a chitosan solution and drying.

Chitosan applied to amino-reactive surfaces using a method of the invention has increased stability since over 3.5 times less chitosan is lost in a water soak treatment. In addition, using a method of the invention, chitosan is stably attached to inert, un-reacted surfaces which have been difficult to use for chitosan attachment.

Polymer Preparation

A polymer to be chitosan coated using a method of the present invention is pretreated such that it acquires a wettable surface if it is not naturally wettable. A wettable surface is one that is hydrophilic to the extent that water does not bead on the surface. One skilled in the art is familiar with different treatments used to produce a wettable surface including, for example, corona treatment, plasma treatment, electrical discharge, acid etching, and various chemical treatments including with organic alcohols such as octanol, heptanol, or hexanol, as well as nonionic, cationic, or anionic surfactants such as di-octyl-sulfo succinate. In addition, a surface may be treated with a polymer to enhance wettability, such as treatment of a polypropylene nonwoven.

In a method of the present invention, no further surface priming treatment is required prior to chitosan treatment. Elimination of the need for surface treatments, such as acid treatment or base and acid treatment, greatly simplifies the process for attaching chitosan to a surface.

Chitosan Attachment

Following the wettable surface preparation, the polymer or polymer-containing article is contacted with chitosan. This comprises soaking or wetting the polymer or polymer-containing article with a chitosan solution to apply a coating. This solution is an aqueous acid solution, typically including about a stoichiometric amount of acid with respect to the concentration of the chitosan. For example, for every gram of chitosan with a degree of deacetylation of 1, 0.375 g of acetic acid provides a stoichiometric amount of acetic acid. Therefore 0.5 g of acetic acid per g of chitosan is typically used. The solution;generally contains chitosan in the range of from about 0.1% to about 10% by weight, preferred is from about 2% to about 10% range, and most preferred is about 4%.

For good wetting and surface coverage, properties of the polymer surface and physical properties of the chitosan solution can be adjusted. The physical properties chosen for the chitosan solution will depend on the substrate to be coated and the coating method. For example, when coating fibrous polymers, it is desirable for the chitosan solution to impregnate the fabric. For this to happen, impregnation generally requires modification of the interfacial tension and the solution viscosity. One way to achieve a low enough interfacial tension in a fabric is by pre-application of a surfactant. Alternatively, a surfactant or alcohol may be added to the chitosan solution to reduce its surface tension. In addition to a low interfacial tension, impregnation of a fabric also requires that the chitosan solution viscosity be low enough to enter the porous structure in the time period allowed. One generally controls the solution viscosity with the choice of chitosan molecular weight and solution concentration.

Coating of a film substrate requires a different set of parameters. Low surface energy is also required, and this is generally achieved by corona treatment of the film surface. The viscosity needed depends on the application method. For example, when coating films by a process using a wire wound rod, the viscosity can be high enough to resist de-wetting, but low enough to flow easily under the rod.

After contact with chitosan, the chitosan-coated polymer is dried by any method commonly known in the art, for example, by vacuum, evaporation (ambient air drying), and air forced drying, each with or without heat, and by oven drying. The chitosan-coated polymer is then rehydrated. Rehydration may be by any means that allows the uptake of water on the surface without washing, such as by spraying the article with water mist or by placing the article in a humidity chamber. For example, the chitosan-coated polymer may be placed in a humidity chamber containing a separate reservoir of water that is not in contact with the polymer. A production process may include spraying a mist onto the polymer, for example, on a rotating drum. Drying and humidification steps may be repeated to promote evaporation of the acid that reformed.

Polymers and articles comprising polymers prepared by a method of the present invention exhibit antimicrobial properties and are expected to inhibit odor development as well. Said antimicrobial properties may, optionally, be further enhanced by treatment with metal salts. Metal salts useful for the present invention include, for example, zinc sulfate, copper sulfate, silver nitrate, or other water-soluble zinc, copper, and silver salts or mixtures of these. The metal salts are typically applied by dipping, spraying, or padding a dilute (0.1% to 5%) solution of the salt in water onto the article. In addition, the metal salt may be included in the chitosan solution used for material treatment.

Applications of Chitosan Coated Materials

Articles comprising the chitosan coated polymeric material of the present invention may be in the form of or comprise a film, membrane, laminate, knit fabric, woven fabric, nonwoven fabric, fiber, filament, yarn, pellet, coating, or foam. Articles may be prepared by any means known in the art, such as, but not limited to, methods of injection molding, extruding, blow molding, thermoforming, solution casting, film blowing, knitting, weaving, spinning, spunbonding, melt blowing, spunlacing, or carding.

The preferred articles of the present invention provide multiple uses, because many articles benefit from a reduction in microbial growth and a wide variety of polymers are included in the present invention. The following are examples of articles where it is desirable to reduce microbial growth in or on the article in the end-use for which the particular article is commonly used.

The articles of the invention include packaging for food, personal care (health and hygiene) items, and cosmetics. By “packaging” is meant either an entire package or a component of a package. Examples of packaging components include, but are not limited to, packaging film, liners, absorbent pads packaging, shrink bags, shrink wrap, trays, tray/container assemblies, caps, adhesives, lids, and applicators. Such absorbent pads, shrink bags, shrink wrap, and trays of the present invention are particularly useful for packaging meat, poultry, and fish. Food packaging is provided an added benefit from the materials and articles of the invention due to the insolubility of the chitosan coating that is achieved in using the method of the invention. The insolubility of any antimicrobial coating is of high importance in food applications so that the coating itself does not leach from the packaging material and become an additive of the food.

The package may be in any form appropriate for the particular application, such as a can, box, bottle, jar, bag, cosmetics package, or closed-ended tube. The packaging may be fashioned by any means known in the art, such as by extrusion, coextrusion, thermoforming, injection molding, lamination, or blow molding.

Some specific examples of packaging include, but are not limited to, bottles, tips, applicators, and caps for prescription and non-prescription capsules and pills; solutions, creams, lotions, powders, shampoos, conditioners, deodorants, antiperspirants, and suspensions for eye, ear, nose, throat, vaginal, urinary tract, rectal, skin, and hair contact; lip product packaging; and caps.

Examples of applicators include lipstick, chapstick, and gloss; packages and applicators for eye cosmetics, such as mascara, eyeliner, shadow, dusting powder, bath powder, blusher, foundation and creams; and pump dispensers and components thereof. These applicators are used to apply substances onto the various surfaces of the body, and reduction of bacterial growth will be beneficial in such applications.

Other forms of packaging components included in the present invention include drink bottle necks, replaceable caps, non-replaceable caps, and dispensing systems; food and beverage delivery systems; baby bottle nipples and caps; and pacifiers. Where a liquid, solution or suspension is intended to be applied, the package may be fashioned for application in a form for dispensing discrete drops or for spraying of droplets. The invention will also find use in pharmaceutical applications fashioned as inhalers.

Examples of end-use applications, other than packaging, in the area of food handling and processing that benefit from antimicrobial functionality and wherein microbial growth is reduced in the particular end-use of the consumer are coatings for components of food handling and processing equipment, such as temporary or permanent food preparation surfaces; conveyer belt assemblies and their components; equipment for mixing, grinding, crushing, rolling, pelletizing, and extruding and components thereof; heat exchangers and their components; drains and their components; equipment for transporting water such as, but not limited to, buckets, tanks, pipes, and tubing; and machines for food cutting and slicing and components thereof. Where the surface of such equipment components is metal, a coating of a polymer containing amino-reactive groups as polymerized could first be applied to the metal surface. Alternatively, a film of such a polymer could be treated with chitosan and then heat sealed to the equipment surface. In one embodiment, the equipment component is a screw for mixing and/or conveying that is an element in a single-screw or twin-screw extruder, such as, but not limited to, an extruder used for food processing; and the polymer coating comprises an ionomer.

Articles of the present invention can also be used in or as items of apparel, such as a swimsuit, undergarment, shoe component (for example, a woven or nonwoven shoe liner or insert), protective sports pad, child's garment, or medical garment (such as a gown, mask, glove, slipper, bootie, or head covering). Such garments particularly benefit from the inhibition of odor development.

Articles of the present invention can also be used in or as medical materials, devices, or implants, such as bandages, adhesives, gauze strips, gauze pads, medical or surgical drapes, syringe holders, catheters, sutures, IV tubing, IV bags, stents, guide wires, prostheses, orthopedic pins, dental materials, pacemakers, heart valves, artificial hearts, knee and hip joint implants, bone cements, vascular grafts, urinary catheter ostomy ports, orthopedic fixtures, pacemaker leads, defibrillator leads, ear canal shunts, cosmetic implants, ENT (ear, nose, throat) implants, staples, implantable pumps, hernia patches, plates, screws, blood bags, external blood pumps, fluid administration systems, heart-lung machines, dialysis equipment, artificial skin, ventricular assist devices, hearing aids, and dental implants.

In the hygiene area, articles of the present invention include personal hygiene garments such as diapers, incontinence pads, panty liners, sanitary napkins, sports pads, tampons and their applicators; and health care materials such as antimicrobial wipes, baby wipes, personal cleansing wipes, cosmetic wipes, diapers, medicated wipes or pads (for example, medicated wipes or pads that contain an antibiotic, a medication to treat acne, a medication to treat hemorrhoids, an anti-itch medication, an anti-inflammatory medication, or an antiseptic).

Articles of the present invention also include items intended for oral contact, such as a baby bottle nipple, pacifier, orthodontic appliance or elastic bands for same, denture material, cup, drinking glass, toothbrush, or teething toy.

Additional child-oriented articles that benefit through comprising the polymeric material of the present invention include baby bottles, baby books, plastic scissors, toys, diaper pails, and a container to hold cleansing wipes.

Household articles of the present invention include telephones and cellular phones; fiberfill, bedding, bed linens, window treatments, carpet, flooring components, foam padding such as mat and rug backings, upholstery components (including foam padding), nonwoven dryer sheets, laundry softener containing sheets, automotive wipes, household cleaning wipes, counter wipes, shower curtains, shower curtain liners, towels, washcloths, dust cloths, mops, table cloths, walls, and counter surfaces.

The current invention is also useful in reducing or preventing biofilm growth on the surface of selective separation membranes (for example, pervaporation, dialysis, reverse osmosis, ultrafiltration, and microfiltration membranes), and air and water filters that comprise polymer with amino-reactive groups, for example, sulfonated aromatic polyamides.

The current invention is also useful in providing an antifouling surface on boat components such as, but not limited to, boat hulls and components thereof, and boat motors and components thereof. A film of a chitosan treated polymer could be heat sealed to the boat component's surface.

Devices used in fluid, e.g., water, transportation and/or storage can also benefit from the antimicrobial polymeric material of the invention. Exemplary devices include, but are not limited to, pipes and tanks. The inner surface, outer surface, or both surfaces of a pipe or tank can comprise an antifouling surface of the invention. If the surface(s) does not comprise a polymer with amino-reactive groups as polymerized, for example, if the surface(s) had a metal surface, a coating of a polymer containing amino-reactive group as polymerized could first be applied to the surface(s). Alternatively, a film of such polymer could be treated with chitosan and then heat sealed to the surface(s).

In order to impart antimicrobial functionality to the products listed, the product can be treated with a chitosan agent according to the method of the invention before it is manufactured, or after, or at any time during manufacture of the product. For example, in making an antimicrobial shower curtain, material having a surface that comprises an effective amount of amino-reactive polymer can be treated according to the method of the invention, followed by fashioning a shower curtain from the treated material. Alternatively, the chitosan treatment may be performed after the material is made into a shower curtain. It is believed that the antimicrobial properties of the material will not change significantly.

### EXAMPLES

The present invention is further defined in the following Examples, in which all parts and percentages are by weight and degrees are Celsius. These Examples, while indicating preferred embodiments of the invention, are given by way of illustration only. From the above discussion and these Examples, one skilled in the art can ascertain the essential characteristics of this invention, and, without departing from the spirit and scope thereof, can make changes and modifications to adapt the invention to various usages and conditions.

The meaning of abbreviations used is as follows: “g” means grams, “μg” means microgram(s), “cm” means centimeter(s), “ml” means milliliter(s), “μl” means microliter(s), “N” means normal, “gsm” means grams per square meter, “min” means minute(s), “cfu” means colony forming units, “Hg” means mercury.

### Example 1

### Adsorption of Chitosan to Polyolefin Film with Inert Surface Without Surface Priming Using Rehydration Treatment

A chitosan preparation, Chitoclear® TM656, was purchased from Primex Corporation (Norway). The Chitoclear® TM656 chitosan had an average molecular weight of 60,000-80,000 Daltons and was more than 85% deacetylated as determined by proton and carbon¹³ NMR spectroscopy. A solution containing 4% chitosan was made by slurrying 30 g of dry chitosan powder in 405 g of water. Then under vigorous agitation, additional water (300 g) mixed with acetic acid (15 g) was added. The solution was stirred for 3 more minutes to yield a smooth syrup-like solution.

A high density polyethylene film, Sclairfilm® with a basis weight of 41 gsm (E. I. du Pont de Nemours and Company, Wilmington, Del.), was corona-treated resulting in a surface energy of approximately 45 dynes/cm. The 4% solution of the high molecular weight chitosan used in this work was suitable for coating the corona treated film. The viscosity was low enough that the coating flowed and leveled on the corona treated film, but it was high enough to resist dewetting before it dried.

Three 12-inch by 19-inch pieces of uncoated film were cut using a template, then each was weighed and a film basis weight was calculated as grams per square meter (gsm) for each piece of film. A six-week-old 4% chitosan solution was coated onto the same 12-inch by 19-inch pieces of film as follows. A film was taped to a glass backing, a 15 ml puddle of chitosan solution was poured on top in front of a #30 wire-wound rod (Paul N. Gardner Co., Pompano Beach, Fla.). The rod was drawn down the polymer film, dragging the puddle of solution in front of it, with excess solution being dragged off the film. The solution was allowed to dry for 24 hours at room temperature. Actual wet coating thickness depends on draw-down speed, coating rheology, and liquid/solid interactions, and so is variable when coating is performed by hand. The coating weight was measured by cutting an 8-inch by 12-inch piece of film from the center of each 12×19 film, and weighing it. Using the basis weight calculated for each uncoated film, the coating weight of each coated film was calculated. Coating weights of the coated films are given in Table 1. All samples for later measurement were taken from this center rectangle.

As a control, one film was placed in a plastic bag, which was then sealed (bag storage, Table 1 sample C-1). A second film was left on the lab bench and misted with a spray bottle of water on seven separate occasions over four days, then placed in a plastic bag which was sealed (water misting, Table 1 sample E-1). The third film was placed in a sealed, 2500-cubic inch Plexiglas chamber at room temperature for 51 hours. Also in this chamber was a tray containing 500 ml water (not in contact with the film) to provide humidity (humidity cabinet, Table 1 sample E-2). A hygrometer inside the chamber registered 85% humidity. After the 51 hours of incubation, the water in the tray in the chamber was measured to have a pH of 5. This resulting pH indicates that the acetic acid associated with the chitosan on the film was volatized during the humidification treatment and was then recondensed into the water in the tray.

To measure attachment of chitosan to the polyethylene film surface, after the 51 hour incubation, six disks with 1.75 inches diameter were punched from each of the three films. Each disk was placed in a container on top of 5.25 ml of de-ionized water with the chitosan-coated side facing down into the water. The film disk floated on top of the water and the water was generally held directly under the film disk by surface tension. The film was removed after two days, and the water in each container was analyzed for chitosan using a colloid titration method (K. Toei and T. Kohara, Anal. Chem. Acta 83 (1975), pp. 59-65). 5 μl of a 0.1% Toluidine blue solution was added to a 5 ml sample of each water soak solution. A solution of 0.025 N potassium polyvinylsulfate (PVSK) was slowly added until the solution turned light pink in color. The amount of PVSK solution added was compared to a calibration curve of PVSK volume vs. chitosan concentration to determine the concentration of chitosan in the water soak solution.

The results were used to calculate the amount of chitosan removed from each film disk during the water soak. The average and standard deviation of the measurements from the set of six disks for each film sample is given in Table 1. The fraction of chitosan removed in this test compared to the original coating thickness is shown on Table 1 as the “fraction of available chitosan removed”.

TABLE 1
Chitosan retention on polyolefin film with varying treatments

| | | Original | Extracted | Fraction of |
|---|---|---|---|---|
| | | chitosan | chitosan per | available |
| | | coating weight | area (std. dev.) | chitosan |
| Sample | Treatment | (gsm) | μg/sq.in. | removed |
| C-1 | Bag storage | 1.38 | 567 (88) | 0.64 |
| E-1 | Water misting | 1.44 | 22 (5) | 0.02 |
| E-2 | Humidity | 1.84 | 4 (1) | 0.003 |
| | cabinet | | | |

These results indicated that the water misting rehydration treatment lead to substantially less chitosan loss from the polyolefin film surface than when no post drying treatment was used; 2% loss vs. 64% loss, respectively. The humidification treatment produced even better chitosan retention, with only a 0.3% loss after soaking.

Transmission Electron Micrographs (TEMs) confirmed that chitosan was retained on the humidity cabinet treated sample surface after water soaking. Chitosan layers were visible on this film, while no chitosan layer was visible on the control C-1 sample.

### Example 2

### Humidity Treatment Provides Improved Chitosan Retention in Presence of Acid

The chitosan coated film samples C-1, E-1, and E-2 of Example 1 were treated with an acid extraction to test the stability of chitosan attachment. A 1.75 inch disk was punched from each sample (without water soak treatment), then cut into quarters and placed in a 22 ml vial with 15 ml of 50% acetic acid. The vials were sonicated at 50° C. for 45 min, and then stirred over night with a magnetic stirrer. The samples were removed from the vials, and the amount of chitosan present in the acid solution was measured by titration as described in Example 1. Duplicates of each measurement were made, and the average and standard deviation are reported in Table 2. The fraction of chitosan removed in this test compared to the original coating thickness is given in Table 2 as the “fraction of available chitosan removed”.

TABLE 2
Acid extraction of chitosan-coated polyethylene film

| | | Original | Extracted | |
|---|---|---|---|---|
| | Original | chitosan | chitosan per | Fraction of |
| | sample/ | coating weight | area (std. dev.) | available |
| Sample | Treatment | (gsm) | μg/sq.in. | chitosan removed |
| C-10 | C-1/bag | 1.38 | 816 (47) | 0.92 |
| | storage | | | |
| C-11 | E-1/water | 1.44 | 1047 (218) | 1.13* |
| | misting | | | |
| E-10 | E-2/ | 1.84 | 618 (16) | 0.52 |
| | humidity | | | |
| | cabinet | | | |

*See Table 5

The results showed that, when sonicated in acetic acid, chitosan was removed from the dried only and the hydrated chitosan-coated film samples. Only the humidity cabinet treated sample retained attached chitosan through this very harsh treatment.

Transmission Electron Micrographs (TEMs) confirmed that chitosan was retained on the humidity cabinet treated sample surface after the acid extraction. Chitosan layers were visible on this film, while no chitosan layer was visible on the other samples.

### Example 3

### Antimicrobial Activity of Chitosan and Humidity Treated Polyolefin Film

Experiments were done to determine whether the humidification treatment of the chitosan coated polyolefin film, which removed acetic acid associated with chitosan on the surface, affected antimicrobial function. A shake-flask test was performed as follows. A single colony of E. coli ATCC # 25922 was used to inoculate 25 ml of Trypticase Soy Broth, which was incubated at 37° C. with shaking overnight. The overnight culture was diluted into sterile phosphate buffer at approximately pH 6.5 to obtain approximately 10⁵ colony forming units per ml (cfu/ml). Secondary dilutions of 10⁻⁴ and 10⁻³ were plated onto Trypticase Soy Agar (TSA) plates in duplicate, incubated at 37° C. overnight, and cfu/ml counts taken.

To a flask containing 50 ml of the phosphate buffer with approximately 9×10⁵ cfu/ml of E. coli was added 0.5 g of humidity treated chitosan on polyolefin film from Example 1. As controls, one flask received 50 ml of phosphate buffer containing no bacteria, one flask received 9×10⁵ cfu/ml alone, and one received 9×10⁵ cfu/ml and 0.5 g of untreated polyolefin film. The flasks were incubated with shaking at room temperature for 8 hours. Dilutions were made and plated onto TSA plates in duplicate, incubated at 37° C. overnight, and cfu/ml counts taken. The sample with chitosan and humidity treated polyolefin film added showed a 3.8 log reduction in cfu/ml as compared to the untreated polyolefin control and a 3.9 log reduction as compared to the bacterial inoculation alone control. This experiment shows a substantial reduction in the number of bacteria present in a sample contacted with material treated with chitosan using the hydration process.

### Example 4

### Improved Adsorption of Chitosan to Amino-Reactive Surface Using Rehydration Treatment

The amino-reactive surface chosen for this evaluation was a Surlyn® ionomer film made from partially sodium-neutralized polyethylene acrylic acid copolymer containing 24% acrylate comonomer. A 4% chitosan solution was made and coated onto the film samples as described in Example 1, except that the chitosan solution was 1 day old. The coating weight was measured to be 3.0 gsm when dry.

As a control, one chitosan-treated film was placed in a plastic bag, which was then sealed (bag storage, Table 2 sample C-3). A second film was placed in a room temperature vacuum oven (25.6 inches Hg vacuum) for 24 hours, then placed in a plastic bag which was sealed (vacuum, Table 2 sample C-4). A third film was left on the lab bench and misted with a spray bottle of water once a day for six days, then placed in a plastic bag which was sealed (water misting, Table 2 sample E-4).

Fifteen days after the coating was applied, disks punched from the films were soaked in water for two days, and the amount of chitosan in the water was measured by titration, all as described in Example 1, to measure attachment of chitosan to the ionomer surface. The amount of chitosan that was removed is summarized in Table 4. The fraction of chitosan removed in this test compared to the original coating thickness is shown on Table 4 as the “fraction of available chitosan removed”.

TABLE 4
Chitosan retention on Surlyn ® ionomer film with varying
treatments

| | | Extracted chitosan per | |
|---|---|---|---|
| | | area | |
| | | (std. dev.) | Fraction of available |
| Sample | Treatment | μg/sq.in. | chitosan extracted |
| C-3 | bag storage | 1503 (94) | .73 |
| C-4 | vacuum oven | 1270 (79) | .65 |
| E-4 | water misting | 354 (209) | .19 |

These results indicated that vacuum oven treatment had a small effect on chitosan retention on the amino-reactive Surlyn® ionomer film surface. However, the water misting rehydration treatment lead to substantially less chitosan loss from the amino-reactive film surface than when no post drying treatment was used: 19% loss vs. 73% loss, respectively.

### Example 5

### Repeated Rehydration and Drying Treatment of Chitosan-Acetic Acid Solution Promotes Insolubility

An organic acid salt solution of chitosan was treated with different drying and rehydration steps to determine whether there were any effects on acid or water solubility. A 4% solution of chitosan (4 weeks old) was pipetted in approximately 15 mg aliquots into 36 separate vials, which were each weighed. Sets of six vials were given 6 different treatments:
1) Samples (C-5) were dried in air for 45 minutes and then further dried in a vacuum oven (25 in Hg) for 30 minutes at 70° C. 20 ml of de-ionized water was then added to each vial, and left closed over night.
2) Samples (C-6) were dried in air for 45 minutes and then further dried in a vacuum oven (25 in Hg) for 30 minutes at 70° C. 20 ml of 1% acetic acid in de-ionized water was then added to each vial, and left closed over night.
3) Samples (E-7) were dried in a 30° C. vacuum oven (25 in Hg) for 30 minutes, taken out, sprayed with water, and returned to the vacuum oven. This was repeated for a total of three sprays and four dryings. 20 ml of de-ionized water was then added to each vial, and left closed over night.
4) Samples (C-7) solution were dried in a 30° C. vacuum oven (25 in Hg) for 30 minutes, taken out, sprayed with water, and returned to the vacuum oven. This was repeated for a total of three sprays and four dryings. 20 ml of 1% acetic acid in de-ionized water was then added to each vial, and left closed over night.
5) Samples (C-8) were capped without drying. 20 ml of de-ionized water was then added to each vial, and left closed over night.
6) Samples (C-9) were capped without drying. 20 ml of 1% acetic acid in de-ionized water was then added to each vial, and left closed over night.

The amount of chitosan in solution was determined for each vial by titration as described in Example 1. Results are given in Table 5.

TABLE 5
Chitosan solutions dried and redissolved to measure effect on
chitosan solubility

| | | | Measured | |
|---|---|---|---|---|
| | | | chitosan | Fraction dissolved |
| | | Redissolved | (std. dev.) | compared with |
| Sample | Drying method | in | (μg) | theoretical value |
| C-5 | Vacuum oven | Water | 737 (51) | 1.10* |
| C-6 | Vacuum oven | Acid | 858 (114) | 1.19 |
| E-7 | Dry/wet/dry 3x | Water | 3.7 (4.5) | 0.005 |
| C-7 | Dry/wet/dry 3x | Acid | 774 (80) | 1.15 |
| C-8 | Never dried | Water | 762 (72) | 1.14 |
| C-9 | Never dried | Acid | 732 (29) | 1.16 |

*The fact that all of the theoretical values are about 15% high reflects a bias, perhaps in weighing the solutions or in solution preparation.

These results showed that the process of repeatedly drying, re-wetting and re-drying a chitosan solution did not affect chitosan solubility in 1% acetic acid but did cause it to become insoluble in water. It is believed that the repeated dry/wet/dry process volatizes the acetic acid associated with the chitosan, thereby inhibiting its solubility in water. These results may explain the improved chitosan attachment to amino-reactive surfaces: some chitosan amine groups are attracted to the amino-reactive surface, and the rest of the chitosan molecules become insoluble on the surface. It may be the insolubility that also provides attachment of chitosan to non-reactive surfaces.

### Example 6

### Aggregate Formation in Aging Chitosan Solutions

Chitosan-acetic acid solutions were found to develop aggregates, which may promote insolubility when acetic acid is removed from chitosan treated surfaces by hydration treatment.

Because chitosan polymer molecular weight characterization based on polymer size, the generally used method, is affected by the electrostatic interactions of the chitosan charges, a method for characterization of molecular weight using light scattering in an aqueous solution with controlled ionic strength was developed. Molecular weight determination from light scattering is based on first principles and is insensitive to shrinkage and expansion effects arising from varying ionic strength. The light scattering profile of fractions eluted from a Size Exclusion Chromatography (SEC) column also was used to determine the presence of aggregates.

The following chitosan solutions that had been aged for a minimum of 2 weeks in a refrigerator at 40° F. (4.4° C.) or in an oven at 50° C. were characterized: 1% chitosan with 1% acetic acid, 2% chitosan with 1% acetic acid, 4% chitosan with 1% acetic acid, and 4% chitosan with 2% acetic acid. Solutions were diluted to 0.15-0.2 mg of chitosan in 200 μl of mobile phase, which was 0.3 M acetic acid and 0.3 M sodium acetate, for liquid chromatography using a Waters Alliance 2690 solvent delivery system, consisting of a pump, autoinjector, and on-line degasser, on Tosoh Bioscience TSK PW columns: either 3000, 4000, or DNA-PW. After exiting the column, the sample flowed through a multi-angle light scattering detector, through a differential viscometer and then through a differential refractive index detector. The light scattering instrument was a Wyatt DSP equipped with a HeNe laser as the light source. The viscosity detector was Viscotek and the differential refractometer was a Waters 410. The flow rate used was 0.5 ml/min, with a column temperature of 30° C.

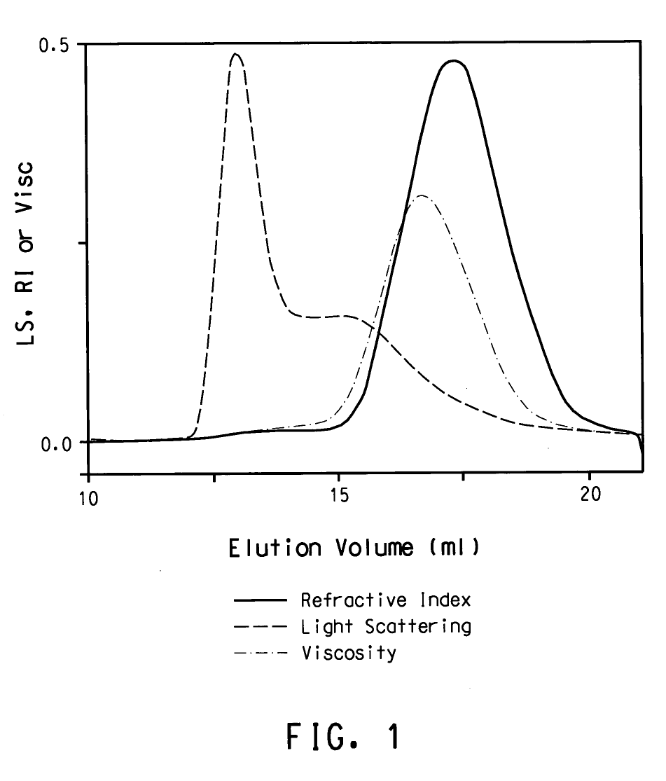

For each sample, chromatograms were obtained from the light scattering, viscosity and refractive index detectors. The refractive index detector measures concentration. The light scattering intensity and the relative viscosity both depend on the product of concentration and a quantity dependent on molecular weight. Light scattering intensity was measured at 15-18 scattering angles. All solutions, except 4% chitosan in 1% acetic acid held at 40° F. (4.4° C.) for two weeks, showed aggregation. These solutions exhibited a complex behavior including some aggregation into multi-molecular species, shrinkage of the solvated chitosan, and significantly reduced intrinsic viscosity for the solutions aged at 50° C. The aggregate peak was not visible in each freshly prepared solution. The aggregate peak represented a very small weight fraction of the sample, but was clearly visible by light scattering. FIG. 1 shows an example of the aggregate peak in the light scattering chromatogram of 4% chitosan in 2% acetic acid held at 50° C. for two weeks. Although the peak is dominant in the light scattering, it is a small fraction of the refractive index and viscosity chromatograms. The aggregates are multi-molecular species of high apparent molecular weight, which scatter light strongly. However, they are denser than solvated chitosan, and contribute little to the viscosity. It is also clear that only a small fraction of the chitosan is aggregated: 2-3wt %. For example, in the sample in FIG. 1, the aggregate peak has an apparent molecular weight over 7 million, but a R_{g} of only 40 nm. An individual solvated chitosan with this molecular weight would be expected to have an Rg of more than 300 nm, or 3 orders of magnitude more volume in solution.

Thus light scattering, viscosity, and refractive index measurements on chitosan fractions eluted from a Size Exclusion Chromatography (SEC) column demonstrated that aggregates of chitosan formed in aged solutions without loss of molecular weight. Not only did these aggregates have a very high molecular weight (indicating that many molecules are involved), but they were also much denser than other solvated chitosan molecules.

1. A method comprising
(a) providing a wettable surface of a polymer;
(b) contacting a chitosan acid salt solution comprising chitosan and at least one volatile organic acid to the wettable surface of (a) to produce a chitosan-coated polymer;
(c) drying, optionally in the presence of heat, the chitosan-coated polymer;
(d) rehydrating the dried chitosan-coated polymer of (c); and
(e) drying the chitosan-coated polymer of (d).
wherein the salt solution is optionally aged for about 2 to about 3 weeks or for about 6 weeks; and (d) and (e) are optionally repeated at least once.

2. The method according to claim 1 wherein the organic acid includes selected from acetic acid, formic acid, butyric acid, proprionic acid, valeric acid, or a mixture thereof.

3. The method according to claim 2 wherein the chitosan acid salt solution comprises the organic acid in a stoichiometric amount with respect to the concentration of the chitosan.

4. The method according to claim 3 wherein chitosan is present in the chitosan acid salt solution in a range of from about 0.1% to about 10% or about 2% to about 10% or at about 4%.

5. The method according to claim 1, wherein the polymer includes polyolefin homopolymers, polyolefin copolymers, polyolefin graft copolymers, polyolefin ionomers, or polyolefin blends.

6. The method according to claim 4, wherein the rehydrating is performed in a humidity chamber and the salt solution is stored at a temperature of about 4.4° C. or about 50° C.

7. The method according to claim 6 wherein the salt solution comprises aggregates or crystals.

8. The method according to claim 1, wherein the polymer comprises amino-reactive functional groups as polymerized and further comprising treating, during or after (b), the polymer with at least one metal salt including a water-soluble zinc salt, a water-soluble copper salt, a water-soluble silver salt, or mixtures thereof.

9. The method according to claim 1 further comprising pretreating, before (a), a nonwettable surface on a polymer to produce a wettable surface on the polymer and the pretreating includes corona treatment, plasma treatment, electrical discharge, acid etching, or chemical treatment.

10. The method according to claim 9 wherein the pretreating is the chemical treatment employing an organic alcohol, a surfactant, or a polymer and the surfactant includes nonionic, cationic, or anionic surfactant.

11. An article comprising a chitosan-coated polymer produced by the method of claim 1 wherein the article includes film, membrane, laminate, knit fabric, woven fabric, nonwoven fabric, fiber, filament, yarn, pellet, coating, or foam.

12. The article of claim 11 having been blown, solution cast, laminated, injection molded, extruded, blow molded, thermoformed, knit, woven, or spun.

13. The article of claim 11 including a package, packaging component, food or beverage dispensing system, baby bottle, baby book, plastic scissors, toy, diaper pail, container for cleansing wipes, baby bottle nipple, pacifier, orthodontic appliance or component thereof, denture material, cup, drinking glass, toothbrush, teething toy, tampon, tampon applicator, personal cleansing wipe, baby wipe, cosmetic wipe, personal hygiene garment, food handling and processing equipment, item of apparel, household article, bandage, adhesive, gauze strip, gauze pad, medical or surgical drape, medical device or implant, separation membrane, air or water filter, boat component, or fluid transportation or storage device wherein
the apparel includes swimsuit, sportswear, active wear, protective sports pad, undergarment, shoe component, child's garment, or medical garment; the shoe component optionally includes a woven or nonwoven liner or insert; and the medical garment optionally includes gown, mask, glove, slipper, bootie, or head covering;
the household article includes fiberfill, bedding, bed linens, window treatments, carpet and flooring components, upholstery components, sheets, automotive wipes, nonwoven dryer sheets, laundry softener-containing sheets, household cleaning wipes, counter wipes, towels, washcloths, dust cloths, mops, tablecloths, shower curtains, telephones and cellular phones, wall surface, counter surface, or floor surface;
the medical device or implant includes syringe holder, catheter, suture, IV tubing, IV bag, stent, guide wire, prostheses, orthopedic pin, dental material, pacemaker, heart valve, artificial heart, knee and hip joint implant, bone cement, vascular graft, bandage, adhesive, gauze strip, gauze pad, urinary catheter ostomy port, orthopedic fixture, pacemaker lead, defibrillator lead, ear canal shunt, cosmetic implant, ENT implant, staple, implantable pump, hernia patch, plate, screw, blood bag, external blood pump, fluid administration system, heart-lung machine, dialysis equipment, artificial skin, ventricular assist device, hearing aid, and dental implant;
the separation membrane includes reverse osmosis, dialysis, pervaporation, ultrafiltration, or microfiltration membrane;
the boat component includes boat hull, a component of a boat hull, a boat motor and a component of a boat motor; and
the fluid transportation or storage device is a pipe or tank.

14. The article of claim 13 wherein the article is the package including bottle, box, jar, can, bag, close-ended tube, cosmetics package, or inhaler and comprises cosmetic, personal hygiene material, healthcare material, or combination thereof and the cosmetic, the personal hygiene material, or the healthcare material includes lipstick, chapstick, eye shadow, eyeliner, mascara, dusting powder, bath powder, blusher, foundation, shampoo, conditioner, deodorant, antiperspirant, lotion, cream, powder, liquid, solution, suspension, capsule, or pill and the package optionally contains a food or a beverage.

15. The article of claim 13 wherein the article is the packaging component including liner, lid, adhesive, replaceable container cap, disposable container cap, film, shrink wrap, shrink bag, tray, tray/container assembly, absorbent pad for packaging, applicator, drink bottle neck, food dispensing system, or beverage dispensing system.

16. The article of claim 13 wherein the article is a packaging for meat, poultry, or fish comprising the shrink wrap, shrink bag, tray, absorbent pad for packaging, and combinations thereof.

17. The article of claim 13 wherein the article is the applicator including pump dispenser or component thereof, mascara wand, medicated pad or wipe, cosmetics brush, dropper, tip, lipstick applicator, eyeliner applicator, or eye shadow applicator.

18. The article of claim 13 wherein the article is the medicated pad or wipe comprising an antibiotic, a medication to treat acne, a medication to treat hemorrhoids, an anti-itch medication, an anti-inflammatory medication, or an antiseptic.

19. The article of claim 13 wherein the article is the personal hygiene garment including a diaper, incontinence garment, panty liner, sanitary napkin, or tampon.

20. The article of claim 13 wherein the article is the food handling and processing equipment including a conveyor belt assembly and components thereof, temporary and permanent food preparation surfaces, equipment for mixing or grinding or crushing or rolling or pelletizing or extruding or components thereof, heat exchanger or its component, drain or its component including bucket or tank or pipe or tubing, or machine for food cutting or slicing or its component thereof; and the equipment for extruding optionally comprises a screw for mixing and/or conveying and wherein the polymer coating comprises an ionomer.
